(19) United States
(12) Patent Application Publication
(10) Pub. No.: US 2024/0148266 A1
(43) Pub. Date: May 9, 2024

(54) Bioimpedance Sensing Devices, Systems, and Techniques to Assess a Fluid State of a Body, or Portion thereof

(71) Applicant: Centrus Diagnostics, Inc, Sammamish, WA (US)

(72) Inventors: Michael H. Burnam, Sammamish, WA (US); Srikanth Jadcherla, Frisco, TX (US)

(21) Appl. No.: 18/493,855

(22) Filed: Oct. 25, 2023

Related U.S. Application Data
(60) Provisional application No. 63/381,403, filed on Oct. 28, 2022.

(51) Int. Cl. A61B 5/0537; A61B 5/00

(52) U.S. Cl. CPC A61B 5/0537; A61B 5/0022; A61B 5/0024; A61B 5/4875; A61B 5/6805; A61B 5/6823; A61B 5/6824; A61B 5/6828; A61B 5/7271; A61B 2560/0214; A61B 2560/045; A61B 2560/0468; A61B 2562/046; A61B 2562/164; A61B 2562/221

(57) ABSTRACT

The present inventions, in one aspect, are directed to bioimpedance sensing devices, systems, and methods, including, for example, utilizing a wearable sensing garment and/or accessory, that sense, acquire, detect and/or measure bioimpedance data to, in one embodiment, calculate, assess, determine and/or monitor data associated with, corresponding to and/or representative of a biological properties (e.g., fluid state or state of hydration) in an animal body (e.g., a human). The wearable sensing garment and/or accessory, having bioimpedance sensors thereon or therein, may be, for example, made of any material now known or later developed and preferably fits tight/snug (selectively or entirely) to the body of the animal so that the bioimpedance sensors make suitable contact to the body to facilitate acquisition of bioimpedance data (e.g., intermittent, periodic, continuous and/or substantially continuous data acquisition). The garment and/or accessory may be a bodysuit, shirt, gloves, pants, sleeves, footwear, belt, band and/or collar.

### RELATED APPLICATION

This non-provisional application claims priority to and the benefit of U.S. Provisional Application No. 63/381,403, entitled “Devices, Systems, and Methods for Assessing Fluid State using Bioimpedance Sensing”, filed Oct. 28, 2022. The '403 provisional application is hereby incorporated herein by reference in its entirety.

### INTRODUCTION

There are many inventions described and illustrated herein. The present inventions are neither limited to any single aspect nor embodiment thereof, nor to any combinations and/or permutations of such aspects and/or embodiments. Importantly, each of the aspects of the present inventions, and/or embodiments thereof, may be employed alone or in combination with one or more of the other aspects of the present inventions and/or embodiments thereof. All combinations and permutations thereof are intended to fall within the scope of the present inventions.

In one aspect, the present inventions are directed to bioimpedance sensing devices, systems, and methods, including, for example, utilizing a wearable sensing garment and/or accessory, that sense, acquire, detect and/or measure bioimpedance data to, in one embodiment, calculate, assess, determine and/or monitor data associated with, corresponding to and/or representative of a biological properties (e.g., fluid state or state of hydration) in an animal body (e.g., a human). The wearable sensing garment and/or accessory includes a plurality of sensors (e.g., bioimpedance sensors such as bioelectric impedance analysis ((BIA) electrodes) to provide data corresponding to the fluid state or state of hydration in/of the body. The wearable sensing garment and/or accessory, having bioimpedance sensors thereon or therein, may be, for example, made of any material now known or later developed and is a wearable garment that is consistent with or correlates to the environment of the situation and/or purpose, function or exercise of the human, such as, for example, a soldier's uniform, a firefighter's protective garb, an athlete's uniform, or a garment to be applied to patients in the field by emergency workers and rescuers, as well as in hospitals and other medical facilities.

Notably, in one embodiment, a wearable sensing garment and accessory is a garment/accessory that is wearable in situ (i.e., worn during normal performance or operation of, for example, a human, and/or worn in the normal/typical environment of performance or operation, for example, a human in connection with the garment). For example, wearable in situ by (i) a soldier during or in performance of combat or the like, (ii) a firefighter during or in performance of firefighting, and/or (iii) an athlete during or in performance of the corresponding sport.

In one embodiment, the wearable sensing garment and/or accessory may be worn or disposed beneath or in conjunction with other or practical clothing (e.g., beneath or in conjunction with a soldier's uniform, a firefighter's protective clothing, and/or an athlete's uniform). Where the garment and/or accessory is worn or disposed beneath other or practical clothing, it may be advantageous to employ one or more light-weight materials. Where the wearable sensing garment and/or accessory is employed or deployed in harsh environments, in one embodiment, such garment and/or accessory may include or be fabricated from a material or fabric that, in hot environments, draws moisture away from the skin of the animal where it may evaporate (i.e., a wicking-type material) and/or, in cold environments, insulates the body of the animal. In addition thereto, or in lieu thereof, in yet another embodiment, the wearable sensing garment and/or accessory may fit tightly or snugly to the body of the animal (e.g., the entire body or selective portion(s) thereof) which may improve contact to the body/skin of the animal to facilitate and/or improve acquisition of bioimpedance data (e.g., intermittent, periodic, continuous and/or substantially continuous data acquisition). Indeed, such a configuration may ensure the bioimpedance sensors make robust/firm/strong and continuous and/or substantially continuous contact with the body/skin of the animal. In one embodiment, the wearable sensing garment and/or accessory may be, for example, a bodysuit (e.g., a garment that fits tightly/snugly to the body of the animal), shirt (e.g., a shirt that fits tightly/snugly to the chest, torso and/or arms of the animal—for example, a compression type shirt), shorts (e.g., compression type), gloves, pants (e.g., compression type), sleeves (e.g., compression type sleeve for the arm(s) and/or leg(s), or portions thereof), footwear (e.g., socks and/or shoes), belt, band (e.g., arm, leg, wrist, ankle, torso and/or abdomen), watch and/or collar.

Indeed, the wearable sensing garment and/or accessory (having bioimpedance sensors) may be a full body suit such as a war fighter's or fire fighter's gear, a vest that would cover regions of the body associated with the thorax, a vest or long sleeve shirt portion with separate anklets garments containing the sensors by the ankles with no sensors in the pant portion, or any other combination or permutation of garment. Thus, the wearable sensing garment may be one contiguous item (e.g., a body suit) or a plurality of discrete items including separate/distinct or integrated garment sections corresponding to one or more portions of the body of the animal—whether the garment sections are to be worn concurrently and/or separately; all combinations or permutations thereof are intended to fall within the scope of the present inventions.

In one embodiment, the bioimpedance sensors may be located in or on the wearable sensing garment and/or accessory such that the sensors directly contact the skin of the animal such that, in operation, the sensors detect, acquire, sense and/or measure a bioimpedance of the body (as a whole) of the animal, and/or one or more particular portions, areas and/or regions of the body of the animal. In addition, in one embodiment, a circuitry module (including circuitry to facilitate operation of the bioimpedance sensing device or system), is disposed on or in the wearable sensing garment and/or accessory (e.g., disposed in a pocket or pouch in/on the wearable sensing garment and/or accessory). In one embodiment, the circuitry module includes receiver circuitry and a processor to receive the bioimpedance data generated by the sensors and determine, assess and/or calculate a fluid state or state of hydration in/of an animal body (or portion(s) thereof) and/or change(s) in the fluid state or state of hydration in/of the body over time (or portion(s) thereof). For example, in one embodiment, the bioimpedance sensors are fixed to particular locations on the sensing garment and/or accessory to contact the skin of the animal to detect, acquire, sense and/or measure bioimpedance data—that is, detect acquire, sense and/or measure bioimpedance data from predetermined, selected and/or particular areas or portions of the body of the animal (e.g., each bioimpedance sensor of the plurality of sensors is disposed in/on particular location of a shirt (e.g., a shirt that fits tight/snug on the chest, abdomen and/or arms (e.g., a compression type shirt) to provide sufficient contact between the sensors and the chest, abdomen and/or arms of the animal). The bioimpedance data from the sensors is provided to a processor (e.g., of the circuitry module) which, using data from one or more, or all of the bioimpedance sensors, assesses, determines and/or monitors a fluid state of the body of the animal, or portion thereof.

Notably, the bioimpedance sensors employed, in one embodiment, include a first electrode configured to contact the animal body (e.g., the skin) and, in operation, output an electrical current (e.g., DC current). A second electrode (of the same and/or a different bioimpedance sensors) is also configured to contact the animal body and, in operation, measures a resultant change in voltage from which an impedance of the body, or portion thereof, may be derived. As the electrical conductivity is different between various bodily tissues (e.g. muscle, fat, bone, etc.) due to their variation in water content, the small electrical current passes through the tissues at different speeds. Having this data, a bioimpedance (magnitude and/or phase) is determined or calculated. The bioimpedance may be employed to determine, calculate and/or estimate body composition, including a fluid state or state of hydration in/of the body, or portion thereof.

In one embodiment, the bioimpedance sensing devices, systems, and methods, implement intermittent, periodic, continuous and/or substantially continuous monitoring of body fluid levels, via bioimpedance sensors disposed in and/or affixed to the garment and/or wearable accessory, so as to determine a fluid state or state of hydration, and/or change therein, of the animal. Moreover, the bioimpedance sensing devices, systems, and methods, may implement real-time or near-real-time (hereinafter collectively “real-time”) monitoring of body fluid levels. For example, the bioimpedance sensors may be located in or on the garment or clothing worn on the body of the animal such that the sensors contact the skin of the animal in order to facilitate, in operation, bioimpedance measurements of the body of the animal, or particular part, area or region of the body of the animal. In one embodiment, the acquisition of bioimpedance data from the sensors is continuous or substantially continuous and provided (via wired or wireless transmission) to the processor in real-time. The processor, using the bioimpedance data, may determine, assess and/or calculate a fluid state in the animal body, or portion thereof, in real time to facilitate monitoring (e.g., intermittent, periodic, continuous and/or substantially continuous) of a fluid state in the entire animal body, or portion thereof (e.g., the chest region or abdomen region). In another embodiment, the processor, using the bioimpedance data, may determine, assess and/or calculate change(s) in the fluid state or state of hydration in/of the body over time (or portion(s) thereof), in real time to facilitate monitoring (e.g., intermittent, periodic, continuous and/or substantially continuous) of a fluid state in the entire animal body, or portion thereof (e.g., the chest region or abdomen region) and/or change(s) in the fluid state or state of hydration in/of the body over time (or portion(s) thereof). Notably, monitoring of a fluid state of the body of an animal may include, for example, in addition to or in lieu of an actual value of a fluid state, (i) monitoring a fluid retention of the animal body (or portion thereof) and/or (ii) detecting whether of a fluid state of the animal body (or portions thereof) is/are within a fluid state range or outside of a fluid state range (e.g., undesirable fluid retention in one or more particular regions of the body, or the entire body of the animal, and/or undesirable fluid deficiency (e.g., dehydration)) in one or more particular regions of the body, or the entire body of the animal.

As intimated above, the present inventions are also directed to bioimpedance sensing devices, systems, and methods that acquire, detect, determine, and/or measure bioimpedance data of one or more portions or regions of an animal (e.g., a human) to, in one embodiment, assess or monitor a fluid state (e.g., a state of hydration) of one or more specific or particular regions or portions of the animal body (e.g., chest, abdomen and/or leg(s) (thigh and/or calf of each/both legs)). For example, in one embodiment, the present inventions may be employed to determine, detect and/or monitor a fluid state or body fluid levels, via bioimpedance sensors disposed in and/or affixed to a chest region and/or the abdomen region of an animal, to detect undesirable fluid retention therein. Fluid retention in the chest region may signal a variety of health conditions, including heart conditions, pulmonary conditions, cardio-pulmonary conditions, whereas fluid retention in the abdomen region may indicate intestinal conditions, swelling, and/or kidney conditions. Here again, the present inventions may employ one or more wearable sensing garments or the like (e.g., bodysuit (e.g., compression type), shirt (e.g., compression type wherein the shirt fits tightly/snugly to the body or portions thereof (e.g., the chest, abdomen and/or arms)) disposed on and/or over, and/or affixed to an animal (e.g., human)—and, in this embodiment, the chest and/or abdomen regions. The bioimpedance sensors may be disposed in or on the garment and/or extremity wear (e.g., accessory) such that the sensors provide sufficient contact to the skin of the animal to facilitate bioimpedance measurements of the body of the animal—for example, in this embodiment, the chest and/or abdomen regions. The data measured by these bioimpedance sensors may be provided to a processor (a local processor via wireless transmission) in real-time (or near-real-time). The processor may evaluate or assess the bioimpedance data to determine and/or calculate a fluid state in chest and/or abdomen regions of the animal body and/or one or more change(s) in the fluid state or state of hydration in the chest and/or abdomen regions over time. In this exemplary embodiment, the bioimpedance sensors may intermittent, periodic and/or continuous acquire the bioimpedance data, which is transmitted in real-time to the processor, which may be configured to monitor, in real time (or near-real-time), a fluid state of the chest and/or abdomen regions of the animal body. As intimated above, the fluid state in the chest and/or abdomen regions of the animal body may signal a variety of health conditions.

Thus, in one embodiment, the present inventions may be employed in connection with devices, systems, and techniques that monitor, measure, determine a bioimpedance of one or more predetermined, selected and/or particular portions or regions of the body of the animal to assess or monitor a fluid state of the body of an animal (e.g., fluid retention) which may be employed to assess a variety of health conditions, including heart conditions, pulmonary conditions, cardio-pulmonary conditions, intestinal conditions, swelling, fluid build-up associated with wounds, insect bites, and the like, and/or kidney conditions.

Traditionally, monitoring a state of hydration has relied on a combination of measured fluid intake, symptoms, and physical signs where practical, and, in some circumstances, on the scene medical measurements such as blood pressure and body temperature. These monitoring techniques are often not practical for various settings, including in the context of applications in which heavy protective clothing and gear (e.g., in the context of fire-fighting, war and/or sporting events) may make taking measurements impractical and under conditions in which other personnel may not be available to make either physical qualitative or quantitative assessment (e.g., during a fire, on a battlefield, on a playing field during a sporting event and/or at geographically remote or distant location).

Moreover, conventional bioimpedance measurement techniques, devices and systems employed to monitor, detect and/or measure a fluid state of an animal body present a variety of issues including, for example, being unable or not configured to measure or detect a fluid state of a particular location, region or area of the animal body. Rather, such conventional bioimpedance techniques, devices and systems detect an overall fluid state of/in the animal body.

In addition, many conventional technologies utilize relatively complex and bulky equipment that is/are not practical or suitable for implementation outside of a medical environment or facility. Often such equipment provides poor measurement quality and, in addition, is not suitable to wear to, for example, facilitate real-time continuous or substantially continuous monitoring of body fluid levels of a user (regardless of the user's activity and/or environment). For example, conventional bioimpedance measurement equipment is/are not suitable to provide continuous and real-time data acquisition, detection and monitoring of a user's bioimpedance and/or transmission thereof to a remote location for external, remote (relative to the user) assessment and/or evaluation of the user's fluid state and, in certain embodiment, implement preventive measures, corrective intervention and/or determination of appropriate treatment.

As such, as noted above, in one aspect, devices, systems, and/or techniques of the present inventions provide intermittent, periodic, continuous and/or substantially continuous bioimpedance sensing so as to provide bioimpedance data to facilitate monitoring of body fluid levels to determine, assess, and/or monitor a fluid state or state of hydration of an animal (e.g., a human) body, or portion(s) thereof. In one embodiment, the present inventions provide accurate sensing and monitoring of such bioimpedance data, via bioimpedance sensors, in a manner that is relatively nonintrusive to the animal subject being monitored and without interfering (e.g., excessively) with the regular activity of the animal (and in some cases more demanding activities in the context of some of the aforementioned applications) of the animal while being monitored. Indeed, the present inventions may reliably and quantitatively assess the fluid and electrolyte balance of tissue of the animal using an external sensing mechanism that may provide continuous (or substantially continuous) monitoring for predetermined and/or extended periods of time and/or may transmit the bioimpedance data in real-time, such as to a remote station for data storage, to implement preventive measures, corrective intervention and/or determination of appropriate treatment.

Additional objects, features, and/or advantages will be set forth in part in the description which follows, and in part will be obvious from the description, or may be learned by practice of this disclosure and/or appendant claims. At least some of these objects and advantages may be realized and attained by the elements and combinations particularly pointed out in the appended claims. It is to be understood that both the foregoing general description and the following detailed description are for example and explanatory only and are not restrictive of the claims; rather the claims should be entitled to their full breadth of scope, including equivalents.

### BRIEF DESCRIPTION OF THE DRAWINGS

The present inventions may be implemented in connection with embodiments illustrated in the attached drawings. These drawings show different aspects of the present inventions and, where appropriate, reference numerals illustrating like structures, components, materials and/or elements in different figures are labeled similarly. It is understood that various combinations of the structures, components, and/or elements, other than those specifically shown, are contemplated and are within the scope of the present inventions.

Moreover, there are many inventions described and illustrated herein. The present inventions are neither limited to any single aspect nor embodiment thereof, nor to any combinations and/or permutations of such aspects and/or embodiments.

Moreover, each of the aspects of the present inventions, and/or embodiments thereof, may be employed alone or in combination with one or more of the other aspects of the present inventions and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s).

The inventions are not limited to the illustrative/exemplary embodiments set forth in this application. Again, there are many inventions described and illustrated herein. The present inventions are neither limited to any single aspect nor embodiment thereof, nor to any combinations and/or permutations of such aspects and/or embodiments. Each of the aspects of the present inventions, and/or embodiments thereof, may be employed alone or in combination with one or more of the other aspects of the present inventions and/or embodiments thereof. For the sake of brevity, many of those combinations and permutations are neither illustrated nor discussed separately herein.

FIG. 1A illustrates, in a schematic diagram form, an exemplary embodiment of a wearable sensing garment, including a plurality of bioimpedance sensors disposed thereon/therein and attached thereto, according to one or more embodiments of the present inventions; the plurality of bioimpedance sensors disposed may be configured in, for example, one or more bioimpedance sensor networks and, in this illustrative embodiment, connected, via wired and/or wireless techniques, to circuitry module, disposed on/in or attached to the garment or an accessory (e.g., belt), to sense, measure and/or detect bioimpedance data to, in operation, calculate, assess, determine and/or monitor data associated with, corresponding to and/or representative of a fluid state or state of hydration in an animal (here, a human) body (or portion thereof) and/or change(s) in the fluid state or state of hydration in/of the body (or portion thereof); in one embodiment, one or more, or all of bioimpedance sensor(s) illustrated represents a plurality of bioimpedance sensors disposed on/in the associated region (e.g., a plurality of bioimpedance sensors (e.g., 2, 3, 4, 5, 6, etc.) located in/on each of leg (112g/112h), each arm (112d/112e), the chest region (112b/112c), abdomen region (112f) and/or neck area (112a)); the electrode(s) of such sensors may be located on one, or more (or all) of the associated region in order to obtain data from different areas of the region;

FIG. 1B illustrates, in block diagram form, an exemplary embodiment of plurality of bioimpedance sensors of a wearable sensing garment of a bioimpedance sensing device wherein each bioimpedance sensor is directly connected to the circuitry module, via wired and/or wireless techniques, according to one or more embodiments of the present inventions; the circuitry module (which may include a processor and power circuitry to, in one embodiment, distribute electrical power to the bioimpedance sensors during operation) is configured to sense, measure and/or detect bioimpedance data to, in operation, calculate, assess, determine and/or monitor data associated with, corresponding to and/or representative of a fluid state or state of hydration in an animal body (or portion thereof) and/or change(s) in the fluid state or state of hydration in/of the body (or portion thereof); the sensors may be configured in one or more bioimpedance sensor networks to provide, in operation, bioimpedance data corresponding to the entire animal body or a portion thereof;

FIG. 2A illustrates, in block diagram form, an exemplary embodiment of plurality of bioimpedance sensors of a wearable sensing garment of a bioimpedance sensing device wherein each bioimpedance sensor is configured to connect directly to a portable and/or wearable electronic device, via wired and/or wireless techniques, according to one or more embodiments of the present inventions; in this illustrative embodiment, the wearable sensing garment may not include a circuitry module disposed therein/thereon; rather the circuitry module, including some or all of the functions and operations thereof, may be performed by circuitry located in the portable and/or wearable electronic device (which may include a processor and power circuitry to distribute power to the bioimpedance sensors during operation); in this illustrative embodiment, the portable and/or wearable electronic device may be configured, in operation, to receive bioimpedance data from the bioimpedance sensors and calculate, assess, determine and/or monitor a fluid state or state of hydration in an animal body (or portion thereof) using the bioimpedance data change(s) in the fluid state or state of hydration in/of the body (or portion thereof); the sensors may be configured in one or more (or all) bioimpedance sensor networks to provide, in operation, bioimpedance data corresponding to the entire animal body or a portion thereof;

FIG. 2B illustrates, in block diagram form, an exemplary embodiment of plurality of bioimpedance sensors of a wearable sensing garment of a bioimpedance sensing device wherein each bioimpedance sensor is configured to connect, via wired and/or wireless techniques, to a circuitry module disposed on or in the garment and/or an accessory coupled to the garment wherein, in this illustrative embodiment, the circuitry module (which may include power circuitry to distribute power to the bioimpedance sensors during operation) may connect, via wired and/or wireless techniques, to a portable and/or wearable electronic device, according to one or more embodiments of the present inventions; the portable and/or wearable electronic device may include a processor which, in operation and in addition to the circuitry module or in lieu thereof, is configured to receive the bioimpedance data to calculate, assess, determine and/or monitor a fluid state or state of hydration in an animal body (or portion thereof) using the bioimpedance data; the sensors may be configured in one or more bioimpedance sensor networks to provide, in operation, bioimpedance data corresponding to the entire animal body or a portion thereof; as noted above, the circuitry module may also include a processor which, in addition to the portable and/or wearable electronic device or in lieu thereof, is configured, in operation, to receive bioimpedance data from the sensors and calculate, assess, determine and/or monitor data associated with, corresponding to and/or representative of a fluid state or state of hydration in an animal body (or portion thereof) and/or change(s) in the fluid state or state of hydration in/of the body (or portion thereof); the circuitry module may be disposed on/in or attached to the garment or an accessory (e.g., belt); where the circuitry module is configured to receive the bioimpedance data to calculate, assess, determine and/or monitor a fluid state or state of hydration (and/or change(s) therein) in an animal body (or portion thereof) using the bioimpedance data from the plurality of sensors, the portable and/or wearable electronic device may receive the fluid state or state of hydration in an animal body from the circuitry module and perform additional data processing and/or assess and/or evaluate the fluid state or state of hydration in an animal and, in certain embodiment, implement preventive measures, corrective intervention and/or determination of appropriate treatment;

FIG. 2C illustrates, in block diagram form, an exemplary embodiment of a wearable sensing garment, having a plurality of bioimpedance sensors, wherein each bioimpedance sensor is configured to connect, via wired and/or wireless techniques, directly to portable and/or wearable electronic device (see, FIG. 2A) and/or to a circuitry module disposed on or in the garment and/or an accessory coupled to the garment which is connect, via wired and/or wireless techniques, directly to portable and/or wearable electronic device (see FIG. 2B), according to one or more embodiments of the present inventions; the portable and/or wearable electronic device in this illustrative embodiment may be configured to receive the bioimpedance data from the sensors, and to calculate, assess, determine and/or monitor a fluid state or state of hydration in an animal body (or portion thereof) using the bioimpedance data and subsequently transmit the fluid state or state of hydration in an animal and/or the bioimpedance data detected by the plurality of sensors, to remote circuitry (e.g., a server and/or storage) in the Internet or remote processing system (e.g., one or more servers in the “cloud” or on the “edge” of the “cloud”—over the air (OTA)); where, however, the wearable sensing garment includes a circuitry module (having a processor) which, in operation, is configured to receive the bioimpedance data from the sensors, and to calculate, assess, determine and/or monitor a fluid state or state of hydration (and/or change(s) therein) in an animal body (or portion thereof) using the bioimpedance data, the portable and/or wearable electronic device may be configured to transmit such fluid state or state of hydration to such remote circuitry (e.g., a server and/or storage) in the Internet or remote processing system; in addition, in certain embodiments, the Internet or remote processing system may transmit data/control corresponding to corrective intervention and/or appropriate treatment to the portable and/or wearable electronic device; in response, the portable and/or wearable electronic device may implement corrective intervention and/or appropriate treatment directly and/or transmit such data/control to the circuitry module of the wearable sensing garment to implement corrective intervention and/or appropriate treatment;

FIG. 2D illustrates, in block diagram form, an exemplary embodiment of plurality of bioimpedance sensors of a wearable sensing garment of a bioimpedance sensing device wherein each bioimpedance sensor is configured to connect, via wired and/or wireless techniques, to a circuitry module disposed on or in the garment and/or an accessory coupled to the garment wherein, in this illustrative embodiment, the circuitry module (which may include power circuitry to distribute power to the bioimpedance sensors during operation) may directly connect, via wired and/or wireless techniques, to remote circuitry (e.g., a server and/or storage) in the Internet or remote processing system (e.g., one or more servers in the “cloud” or on the “edge” of the “cloud”—over the air (OTA)), according to one or more embodiments of the present inventions; in one embodiment, the wearable sensing garment includes a circuitry module, having a processor, which, in operation, is configured to receive the bioimpedance data from the sensors, and to calculate, assess, determine and/or monitor a fluid state or state of hydration in an animal body (or portion thereof) using the bioimpedance data, and may be configured to transmit such fluid state or state of hydration to such remote circuitry (e.g., a server and/or storage) in the Internet or remote processing system; in addition thereto, or in lieu thereof, the circuitry module may transmit the bioimpedance data to such remote circuitry (e.g., a server and/or storage) in the Internet or remote processing system to calculate, assess, determine and/or monitor a fluid state or state of hydration in an animal body (or portion thereof) using the bioimpedance data and/or change(s) in the fluid state or state of hydration in/of the body (or portion thereof); moreover, in certain embodiments, the Internet or remote processing system may transmit data/control corresponding to preventive measures, corrective intervention and/or determination of appropriate treatment to the circuitry module, which, in response, may implement corrective intervention and/or appropriate treatment;

FIG. 3A illustrates, in block diagram form, an exemplary embodiment, of the circuitry module disposed on or in the wearable sensing garment and/or accessory coupled thereto, of a bioimpedance sensing device or system, according to one or more embodiments of the present inventions; in one embodiment, the circuitry module is disposed in a pocket in/on the wearable sensing garment and/or accessory; the pocket may include a closing mechanism, such as a zipper, a button, Velcro, or other similar mechanism to close the pocket and thereby secure the circuitry module in a relatively fixed location in/on the wearable sensing garment and/or accessory; the circuitry module may be located locally (on or substantially on the animal via disposed on or in the wearable sensing garment and/or accessory) or remotely;

FIG. 3B illustrates, in block diagram form, an exemplary embodiment of exemplary functions, elements and/or operations, of the circuitry module illustrated in FIG. 3A, according to one or more embodiments of the present inventions;

FIG. 3C illustrates, in block diagram form, an exemplary embodiment of a bioimpedance sensor including power circuitry (e.g., battery) resident therein, according to one or more embodiments of the present inventions; notably, this embodiment of a bioimpedance sensor may be employed in any of the embodiments described and/or illustrated herein;

FIG. 3D illustrates, in block diagram form, an exemplary embodiment of a bioimpedance sensor including power circuitry (e.g., battery), resident therein, which is provides power to one or more associated bioimpedance sensors, according to one or more embodiments of the present inventions; here, the bioimpedance sensor having power circuitry (labeled 112i in FIG. 3D) functions as a power supply hub for the one or more associated bioimpedance sensors (labeled 112x . . . in FIG. 3D); notably, this bioimpedance sensor having a power supply hub may be employed in any of the embodiments described and/or illustrated herein; and

FIGS. 4A-4G illustrate, in block diagram form, exemplary embodiments of sensor networks of the wearable sensing garment including different exemplary combinations of bioimpedance sensors, attached thereto or therein, that form the sensor networks, according to certain aspects of the present inventions; the bioimpedance sensor numbers (112x, x=a to h) in each illustrative embodiment is in relation or reference to the number nomenclature of the bioimpedance sensor locations of the wearable sensing garment illustrated in FIG. 1A; in one embodiment, the processor (resident/local and/or remote) may intermittently, periodically, continuously and/or substantially continuously monitoring of body fluid levels, using impedance measured by the bioimpedance sensors of each sensor subnetwork separately, so as to determine a fluid state or state of hydration, and/or change therein, of the body of the animal based on the bioimpedance sensors of each sensor subnetwork separately; wherein the fluid state or state of hydration, and/or change therein, using the impedance data measured by only the bioimpedance sensors of a given subnetwork corresponds to the portion of the body contacted by the bioimpedance sensors of that sensor subnetwork; in one embodiment, the bioimpedance sensors of the sensor network and subnetworks are organized and arranged according to regions of the body—and the body of the user may be viewed as being compartmentalized or segmented, from a data acquisition and analysis perspective; in these exemplary embodiments, the data processing implemented by the processor (whether the processors is local or remote) may organize data processing on a subnetwork basis (all of the sensors of a given network) or on a network basis (all of the sensors in the system) to, for example, improve and/or focus determination, calculation and/or monitoring of biological properties (e.g., fluid state or state of hydration, or changes therein) of selected regions of the body in/of a user with greater accuracy.

Again, there are many inventions described and illustrated herein. The present inventions are neither limited to any single aspect nor embodiment thereof, nor to any combinations and/or permutations of such aspects and/or embodiments. Each of the aspects of the present inventions, and/or embodiments thereof, may be employed alone or in combination with one or more of the other aspects of the present inventions and/or embodiments thereof. For the sake of brevity, many of those combinations and permutations are not discussed or illustrated separately herein.

### DETAILED DESCRIPTION

In one aspect, the present inventions are directed to bioimpedance sensing devices, systems, and methods, including, for example, utilizing a wearable sensing garment and/or accessory, that sense, acquire, detect and/or measure bioimpedance data to, in one embodiment, calculate, assess, determine and/or monitor data associated with, corresponding to and/or representative of a biological properties (e.g., fluid state or state of hydration, and/or change(s) in the fluid state or state of hydration in/of the body (or portion(s) thereof)) in an animal body (e.g., a human). The wearable sensing garment and/or accessory includes a plurality of sensors (e.g., bioimpedance sensors such as bioelectric impedance analysis ((BIA) electrodes) to provide data corresponding to the fluid state or state of hydration in/of the body. The wearable sensing garment and/or accessory, having bioimpedance sensors thereon or therein, may be, for example, made of any material now known or later developed and is a wearable garment that is consistent with or correlates to the environment of the situation and/or purpose, function or exercise of the human, such as, for example, a soldier's uniform, a firefighter's protective garb, an athlete's uniform, or a garment to be applied to patients in the field by emergency workers and rescuers, as well as in hospitals and other medical facilities.

In one embodiment, a wearable sensing garment and accessory is a garment/accessory that is wearable in situ (i.e., worn during normal performance or operation of, for example, a human, and/or worn in the environment of normal performance or operation, for example, a human). For example, wearable in situ by (i) a soldier during or in performance of combat or the like, (ii) a firefighter during or in performance of firefighting, and/or (iii) an athlete during or in performance of the corresponding sport.

The wearable sensing garment and/or accessory may be worn or disposed beneath or in conjunction with other or practical clothing (e.g., beneath or in conjunction with a soldier's uniform, a firefighter's protective clothing, and/or an athlete's uniform). Where the garment and/or accessory is worn or disposed beneath other or practical clothing, it may be advantageous to employ light-weight materials. Where the wearable sensing garment and/or accessory is employed or deployed in harsh environments, in one embodiment, such garment and/or accessory may include or be fabricated from a material or fabric that, in hot environments, draws moisture away from the skin of the animal where it may evaporate (i.e., a wicking-type material) and/or, in cold environments, insulates the body of the animal. In addition thereto, or in lieu thereof, in yet another embodiment, the wearable sensing garment and/or accessory may fit tightly or snugly to the body of the animal (e.g., the entire body or selective portion(s) thereof) which may improve contact to the body/skin of the animal to facilitate and/or improve acquisition of bioimpedance data (e.g., intermittent, periodic, continuous and/or substantially continuous data acquisition). Indeed, such a configuration may ensure the bioimpedance sensors make robust/firm/strong and continuous and/or substantially continuous contact with the body/skin of the animal. In one embodiment, the wearable sensing garment and/or accessory may be, for example, a bodysuit (e.g., a garment that fits tightly/snugly to the body of the animal), shirt (e.g., a shirt that fits tightly/snugly to the chest, torso and/or arms of the animal—for example, a compression type shirt), shorts (e.g., compression type), gloves, pants (e.g., compression type), sleeves (e.g., compression type sleeve for the arm(s) and/or leg(s), or portions thereof), footwear (e.g., socks and/or shoes), belt, band (e.g., arm, leg, wrist, ankle, torso and/or abdomen), watch and/or collar.

Indeed, in one embodiment, the wearable sensing garment and/or accessory may be a full body suit such as a war fighter's or fire fighter's gear, a vest with sensors that would cover regions of the body associated with the thorax, a vest or long sleeve shirt portion with separate anklets garments containing the sensors by the ankles instead of the pant portion, or a variety of other combinations of garment. Notably, the wearable sensing garment may be one item or more than one item including separate or integrated garment sections to be worn concurrently or separately.

Notably, in one embodiment, one or more bioimpedance sensors may be directly attached to the skin of a body of the user. For example, bioimpedance sensors may be attached directly to the skin of the body via adhesive, medical tape, or a lightweight harness that strings together one or more bioimpedance sensors, or a combination of any of the above. These embodiments may be employed in lieu of a wearable sensing garment or accessory (i.e., one or more wearable accessories, such as a belt, ring, watch, necklace, collar, bracelet, etc.), or in addition thereto. All combinations and permutations are intended to fall within the scope of the present inventions.

Where the bioimpedance sensors are located in or on the garment and/or accessory, the sensors are configured to directly contact the skin of the animal such that, in operation, the sensors detect, acquire, sense and/or measure a bioimpedance of the body (as a whole) of the animal, and/or one or more particular portions, areas and/or regions of the body of the animal. In addition, in one embodiment, a circuitry module (including circuitry to facilitate operation of the bioimpedance sensing device or system), is disposed on or in the wearable sensing garment and/or accessory (e.g., disposed in a pocket or pouch in/on the wearable sensing garment and/or accessory). In one embodiment, the circuitry module includes receiver circuitry and a processor to receive the bioimpedance data generated by the sensors and determine, assess and/or calculate a fluid state or state of hydration in/of an animal body, or portion thereof and/or change(s) in the fluid state or state of hydration in/of the body (or portion thereof). For example, in one embodiment, the bioimpedance sensors are fixed to particular locations on the sensing garment and/or accessory to contact the skin of the animal to detect, acquire, sense and/or measure bioimpedance data—that is, detect acquire, sense and/or measure bioimpedance data from predetermined, selected and/or particular areas or portions of the body of the animal (e.g., each bioimpedance sensor of the plurality of sensors is disposed in/on particular location of a shirt (e.g., a shirt that fits tight or snug on the chest, abdomen and/or arms (e.g., a compression type shirt) to provide sufficient contact between the sensors and the chest, abdomen and/or arms of the body of the animal). The bioimpedance data from the sensors is provided to a processor (e.g., of the circuitry module) which, using data from one or more, or all of the bioimpedance sensors, assesses, determines and/or monitors a fluid state of the body of the animal, or portion thereof.

In one embodiment, the circuitry module is connected (wired or wirelessly) to the bioimpedance sensors to, among other things, control the sensors (e.g., control the acquisition of bioimpedance data by the sensors) and acquire or receive bioimpedance data therefrom. The control module, and specifically the processors, performs processing algorithms determine, assess and/or calculate a fluid state or state of hydration (and/or change(s) therein) in/of an animal body, or portion thereof, regardless of motion of the body during signal acquisition. In one embodiment, the circuitry module employs low power circuits and techniques processing to improve and/or provide power consumption including reduced battery weight providing longer life. Further the ability to sense bioimpedance and monitor for conditions may be via a continuous, real-time data stream, rather than discrete measurements taken intermittently or upon symptomatic episodes. To this end, in one embodiment, the bioimpedance sensing devices, systems, and methods employ artificial intelligence/machine learning (“AI/ML”) subroutines programmed into the circuitry module to manage, reduce and/or minimize intermittent loss of contact by one or more sensors with respect to the body of the animal.

Briefly, the bioimpedance sensors that are integrated into or onto the garment and/or accessory into wearable applications (e.g., in situ applications), in one or more embodiments of the present inventions, include an electrode configured to contact the animal body (e.g., the skin) and, in operation, outputs an electrical current (e.g., DC current). Electrodes of the same bioimpedance sensor and/or different bioimpedance sensor(s) may measure a resultant change in voltage from which an impedance of the body, or portion thereof, may be derived. In one embodiment, the processor in the circuitry module employs a measurement of electrical impedance (magnitude and/or phase) across a volume of tissue of the animal body to assess the fluid and electrolyte balance of the tissue, which may be employed to assess and/or monitor a fluid state (e.g., dehydration) in the animal body. Notably, any technique now known or later developed to assess a fluid state and/or a balance of the fluid to electrolyte in the tissue of the body may be employed to assess and/or monitor a fluid state (e.g., dehydration) in the animal body and is intended to fall within the scope of the present inventions.

In one embodiment, the bioimpedance sensing devices, systems, and methods, implement intermittent, periodic, continuous and/or substantially continuous monitoring of body fluid levels, via bioimpedance sensors disposed in and/or affixed to the garment and/or wearable accessory, so as to determine a fluid state or state of hydration of the animal. Moreover, the bioimpedance sensing devices, systems, and methods, may implement real-time or near-real-time (hereinafter collectively “real-time”) monitoring of body fluid levels. For example, the bioimpedance sensors may be located in or on the garment or clothing worn on the body of the animal such that the sensors contact the skin of the animal in order to facilitate, in operation, bioimpedance measurements of the body of the animal, or particular part, area or region of the body of the animal. In one embodiment, the acquisition of bioimpedance data from the sensors is continuous or substantially continuous and provided (via wired or wireless transmission) to the processor in real-time. The processor, using the bioimpedance data, may determine, assess and/or calculate a fluid state and/or change in fluid state in the animal body, or portion thereof, in real time to facilitate monitoring (e.g., intermittent, periodic, continuous and/or substantially continuous) of a fluid state in the entire animal body, or portion thereof (e.g., the chest region or abdomen region).

Notably, monitoring of a fluid state of the body of an animal may include, for example, in addition to or in lieu of an actual value of a fluid state, (i) monitoring a fluid retention of the animal body (or portion thereof) and/or (ii) detecting whether of a fluid state of the animal body (or portions thereof) is/are within a fluid state range or outside of a fluid state range (e.g., undesirable fluid retention in one or more particular regions of the body, or the entire body of the animal, and/or undesirable fluid deficiency (e.g., dehydration)) in one or more particular regions of the body, or the entire body of the animal.

As intimated above, the present inventions are also directed to bioimpedance sensing devices, systems, and methods that acquire, detect, determine, and/or measure bioimpedance data of one or more portions or regions of an animal (e.g., a human) to, in one embodiment, assess or monitor a fluid state (e.g., a state of hydration) of one or more specific or particular regions or portions of the animal body (e.g., chest, abdomen and/or leg(s) (thigh and/or calf of each/both legs)). For example, in one embodiment, the present inventions may be employed to determine, detect and/or monitor a fluid state or body fluid levels, via bioimpedance sensors disposed in and/or affixed to a chest region and/or the abdomen region of an animal, to detect undesirable fluid retention therein. Fluid retention in the chest region may signal a variety of health conditions, including heart conditions, pulmonary conditions, cardio-pulmonary conditions, whereas fluid retention in the abdomen region may indicate intestinal conditions, swelling, and/or kidney conditions. Here again, the present inventions may employ one or more wearable sensing garments or the like (e.g., bodysuit (e.g., compression type), shirt (e.g., compression type wherein the shirt fits tightly to the body or portions thereof (e.g., the chest, abdomen and/or arms)) disposed on and/or over, and/or affixed to an animal (e.g., human)—and, in this embodiment, the chest and/or abdomen regions. The bioimpedance sensors may be disposed in or on the garment and/or extremity wear (e.g., accessory) such that the sensors provide sufficient contact to the skin of the animal to facilitate bioimpedance measurements of the body of the animal—for example, in this embodiment, the chest and/or abdomen regions. The data measured by these bioimpedance sensors may be provided to a processor (a local processor via wireless transmission) in real-time (or near-real-time). The processor may evaluate or assess the bioimpedance data to determine and/or calculate a fluid state (or change in fluid state) in chest and/or abdomen regions of the animal body. In this exemplary embodiment, the bioimpedance sensors may intermittent, periodic and/or continuous acquire the bioimpedance data, which is transmitted in real-time to the processor, which may be configured to monitor, in real time (or near-real-time), a fluid state of the chest and/or abdomen regions of the animal body. As intimated above, the fluid state in the chest and/or abdomen regions of the animal body may signal a variety of health conditions.

In another aspect, in one embodiment, the present inventions may be employed in connection with devices, systems, and techniques that monitor, measure, determine a bioimpedance of one or more predetermined, selected and/or particular portions or regions of the body of the animal to assess or monitor a fluid state of the body of an animal (e.g., fluid retention), and/or one or more predetermined, selected and/or particular portions or regions of the body. Such data may be employed to measure, assess and/or monitor, in addition to or in lieu of fluid state or state of hydration of the body of the animal, a variety of health conditions, including heart conditions, pulmonary conditions, cardio-pulmonary conditions, intestinal conditions, swelling, fluid build-up associated with wounds, insect bites, and the like, and/or kidney conditions.

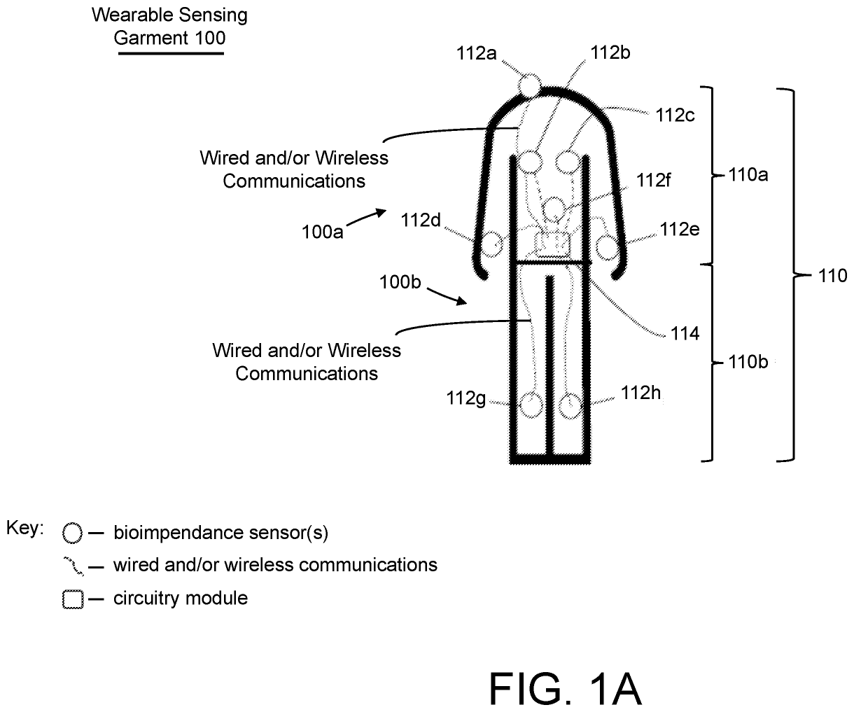

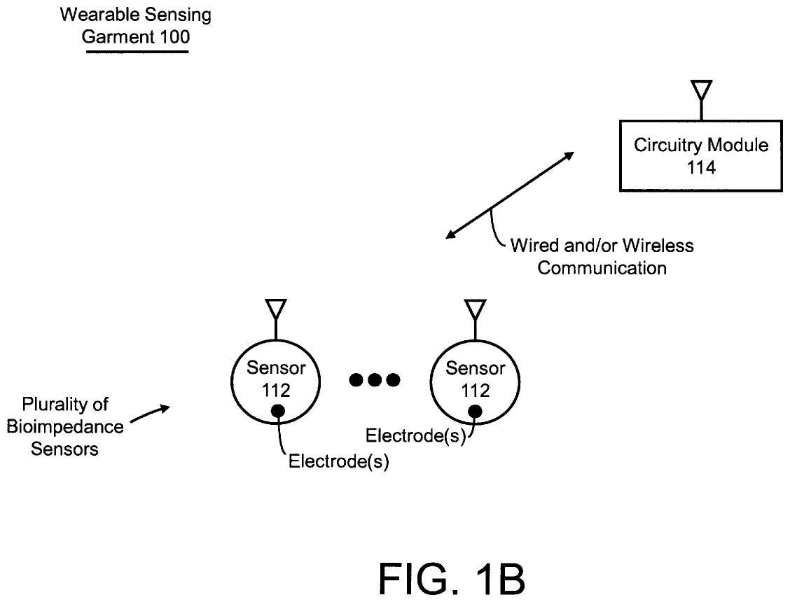

With reference to FIGS. 1A and 1B, in one embodiment, a wearable sensing garment 100 includes a plurality of bioimpedance sensors 112, each sensor having one or more sensing electrodes (e.g., silver/silver chloride gel electrodes and/or solid state polymeric electrodes). In operation, wearable sensing garment 100 is to be worn by a user (i.e., an animal, e.g., a human) in situ—that is, worn during typical/normal performance or operation of a user's routine, and/or worn in the typical/normal environment during typical/normal performance or operation of a user's routine. In one embodiment, one or more, or all of bioimpedance sensor(s) illustrated represents a plurality of bioimpedance sensors disposed on/in the associated region (e.g., a plurality of bioimpedance sensors (e.g., 2, 3, 4, 5, 6, etc.) located in/on each of leg (112g/112h), each arm (112d/112e), the chest region (112b/112c), abdomen region (112f) and/or neck area (112a)). Notably, the electrode(s) of such sensors may be located on one, or more (or all) sides of the associated region in order to obtain data from different areas of the region.

In one embodiment, bioimpedance sensors 112 are arranged or configured in one or more sensor networks 110. For example, all of bioimpedance sensors 112 of wearable sensing garment 100 are arranged in one bioimpedance sensor network 110. In another embodiment, the sensor network(s) may be further arranged or configured into a plurality of subnets (see, e.g., sensor subnets 110a and 110b in the illustrative embodiment). For example, all of bioimpedance sensors 112 of the upper torso (including the arms—(i.e., sensors 112d/112e, 112b/112c, 112f, and 112a—each of which may be a plurality of bioimpedance sensors (e.g., 2, 3, 4, 5, 6, etc.))) are arranged or configured in bioimpedance sensor network 110a; and all of bioimpedance sensors 112 of the lower extremities of wearable sensing garment 100 (i.e., 112g and 112h—each of which may be a plurality of bioimpedance sensors (e.g., 2, 3, 4, 5, 6, etc.)) are arranged in bioimpedance sensor network 110b.

With continued reference to FIG. 1A, the electrode(s) of each bioimpedance sensor 112 may be fixed or fastened to/on wearable sensing garment 100 via an adhesive or the like and arranged to/on garment 100 at predetermined or selected locations. For example, the plurality of sensing electrodes in this illustrative embodiment are positioned and/or fixed at locations in wearable sensing garment 100 corresponding to various locations of the body of the user when garment 100 is worn by the user during typical/normal performance or operation of a user's routine, and/or worn in the typical/normal environment during typical/normal performance or operation of a user's routine (i.e., in situ). For example, in one embodiment, wearable sensing garment 100 includes bioimpedance sensors 112a-112h (each sensor including at least one sensing electrode), arranged in sensor network 110 and located in/on wearable sensing garment 100, as illustrated—including bioimpedance sensor 112a at a collar region of garment 100, bioimpedance sensor 112b, 112c at opposite lateral sides of the chest area, bioimpedance sensor 112d, 112e at each of the sleeves, sensing bioimpedance sensor 112f at waist, abdomen or belt line region of garment 100, and sensing electrode 112g, 112h at each of the leg or ankle regions of garment 100. Notably, in this illustrative embodiment, wearable garment 100 includes a long sleeve shirt portion 100a and long pant portion 100b, wherein (i) each of portion of garment 100a and garment 100b may be separate portions of garments or a single, unitary garment and/or (ii) garment 100a and 100b may be separate portions of garment 100 or may be a single, unitary garment 100.

With reference to FIGS. 1A and 1B, regardless of the construction or structural configuration of garment 100, however, bioimpedance sensor 112a-112h (each sensor including one or more sensor electrodes) are communicatively coupled to a circuitry module 114 (see, e.g., dashed lines from bioimpedance sensor 112a-112h to circuitry module 114 in FIG. 1A) worn to, in one embodiment, supply power to, control, and transmit data from bioimpedance sensors 112a-112h. The bioimpedance sensor 112a-112h may be communicatively coupled to circuitry module 114 in wireless or wired manner via electrical and data transmission lines (e.g., cables or wires) or in a wireless manner (e.g., low power Bluetooth or the like), or a combination of both wired and wireless connection (e.g., power is provided to bioimpedance sensors 112a-112h via wired techniques and data/control is provided between sensors 112a-112h via wireless techniques). Where the communication between circuitry module 114 and sensors 112a-112h employs a wired technique, in an embodiment, the signal lines may be insulated cabled fixed in/on (e.g., sewn or pressed into) wearable garment 100.

The circuitry module 114 may be disposed on or in and/or affixed (e.g., temporarily) to wearable garment 100, for example, in a pocket or pouch (not illustrated) of wearable garment, wherein circuitry module 114 may securely connect to bioimpedance sensors 112a-112h. In one embodiment, the pocket or pouch may include a mechanism to securely maintain circuitry module 114 in or to garment, such as, for example, a zipper, a button, Velcro, or similar mechanism to close the pocket/pouch to prevent the circuitry module 114 dislodging or escaping from the pocket/pouch of the garment. The pocket/pouch may be integrated into as part of shirt portion 100a or pant portion 100b of garment 100 or be included as part of a belt worn in conjunction with garment 100 or secured to garment 100.

Notably, bioimpedance sensors 112a-112h may be configured to detect a range of electrical resistance, which may be set, programmed (one time or more than one time) and/or modified (one time or more than one time) by, for example, circuitry module 114. The circuitry module 114 may be programmed or detect user environment or operation and, based on the particular body and application of the user, set, program (one time or more than one time) and/or modify (one time or more than one time) such range.

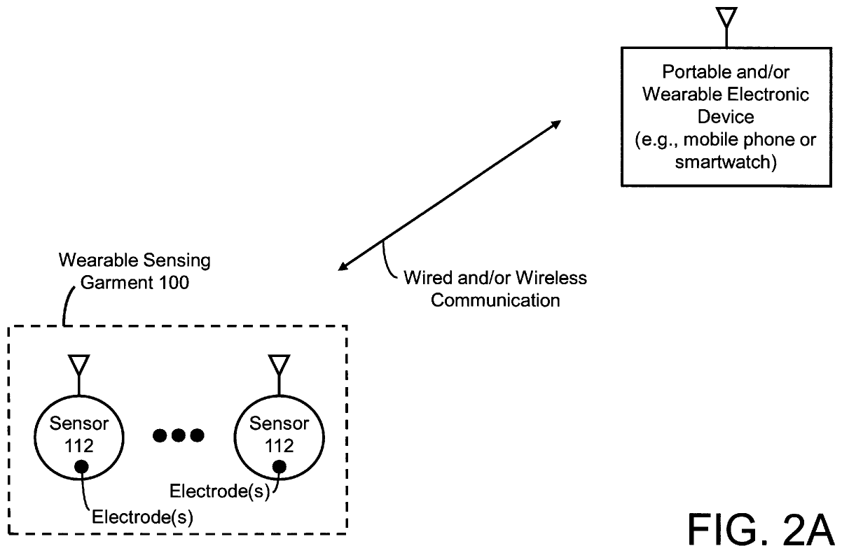

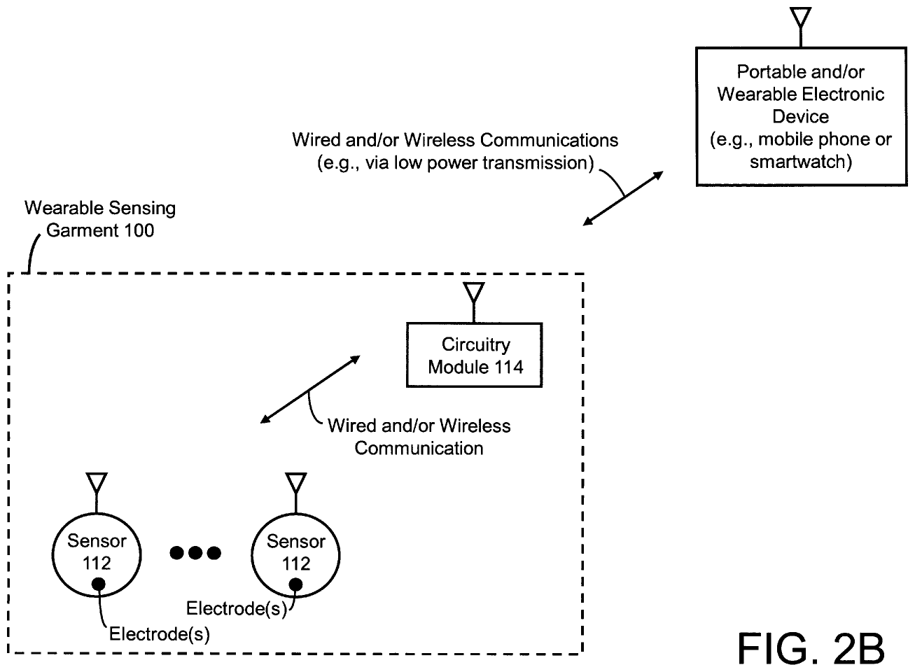

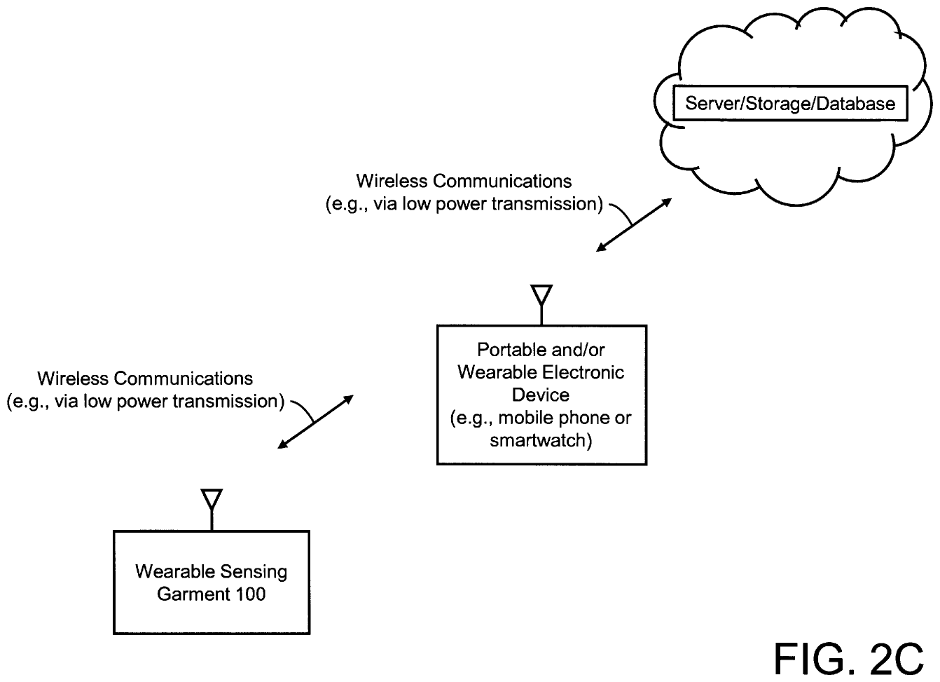

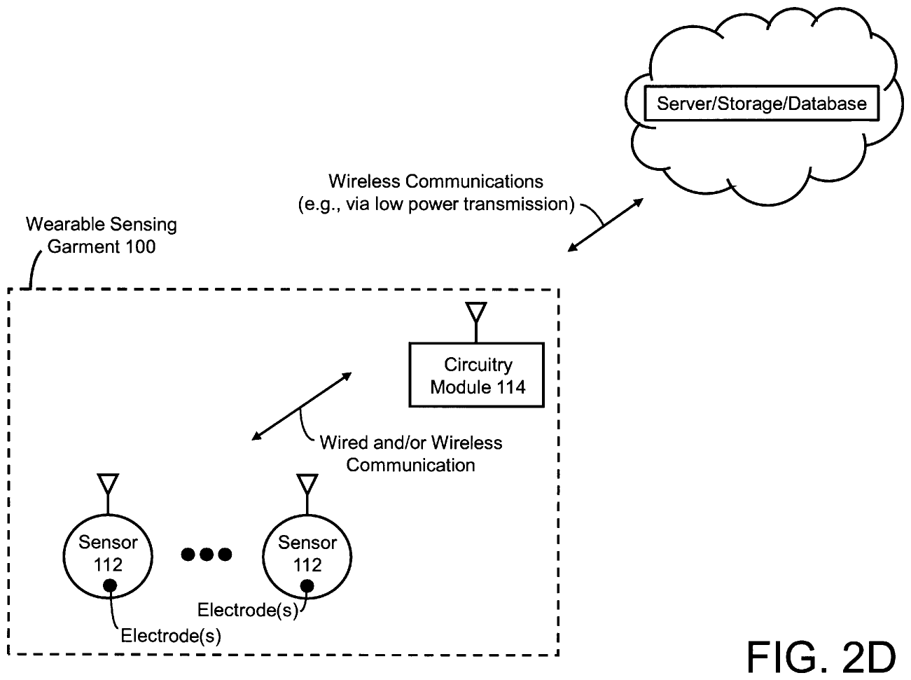

With reference to FIG. 2A, in one embodiment, the plurality of bioimpedance sensors (each including one or more electrodes) of a wearable sensing garment may be directly connected to a portable and/or wearable electronic device, via wired and/or wireless techniques. In another embodiment, the plurality of bioimpedance sensors (each including one or more electrodes) of a wearable sensing garment may be directly connected to circuitry module 114 (see, e.g. FIG. 1B), which is directly connected to a portable and/or wearable electronic device, via wired and/or wireless techniques. (See, FIGS. 2B and 2C). In the illustrative embodiments of FIGS. 2A to 2C, the wearable sensing garment may or may not include a circuitry module disposed therein/thereon. Rather the circuitry module, including some or all of the functions and operations thereof, may be performed by circuitry in the portable and/or wearable electronic device (which may include a processor and power circuitry to distribute power to the bioimpedance sensors during operation) in addition to the circuitry module or in lieu thereof. Thus, in these illustrative embodiments, the portable and/or wearable electronic device may be configured, in operation, to receive bioimpedance data from the bioimpedance sensors and calculate, assess, determine and/or monitor a fluid state or state of hydration in an animal body (or portion thereof) using the bioimpedance data change(s) in the fluid state or state of hydration in/of the body (or portion thereof).

Notably, the embodiments of FIGS. 2A to 2C may be implemented in connection with other embodiments described and/or illustrated herein, for example, in relation to the garment configuration/material, sensor location(s) and configuration(s). Moreover, the bioimpedance sensors may be configured in one or more bioimpedance sensor networks to provide, in operation, bioimpedance data corresponding to the entire animal body or a portion thereof. Indeed, for the avoidance of doubt, the embodiment including the portable and/or wearable electronic device may be employed in any of the embodiments described and/or illustrated herein.

With reference to FIGS. 2C and 2D, in one embodiment, the portable and/or wearable electronic device (FIG. 2C) and/or the circuitry module (FIG. 2D) may be configured to receive the data from the bioimpedance sensors, and calculate, assess, determine and/or monitor a fluid state or state of hydration in an animal body (or portion thereof) using the bioimpedance data and thereafter transmit the fluid state or state of hydration (or change therein) in an animal and/or the bioimpedance data detected by the plurality of sensors, to remote circuitry (e.g., a server and/or storage) in the Internet or remote processing system (e.g., one or more servers in the “cloud” or on the “edge” of the “cloud”—over the air (OTA)). The remote circuitry or processing system may further analyze the fluid state or state of hydration (or change therein) as well as further analyze the “raw” bioimpedance data from the sensors. In response, the remote circuitry or processing system may transmit data/control corresponding to corrective intervention and/or appropriate treatment, if any, to the portable and/or wearable electronic device (FIG. 2C) and/or the circuitry module (FIG. 2D). In response, the portable and/or wearable electronic device may implement corrective intervention and/or appropriate treatment directly and/or transmit such data/control to the circuitry module of the wearable sensing garment to implement corrective intervention and/or appropriate treatment. Where the circuitry module directly connects to the remote circuitry or processing system, the circuitry module may receive the data/control corresponding to corrective intervention and/or appropriate treatment directly and, in response, implement corrective intervention and/or appropriate treatment directly.

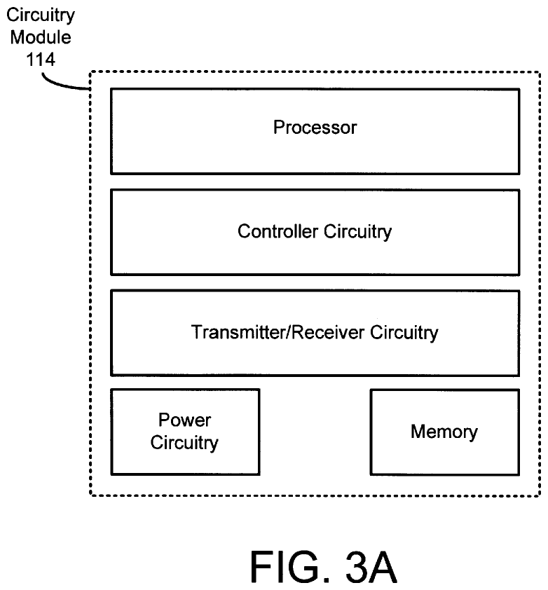

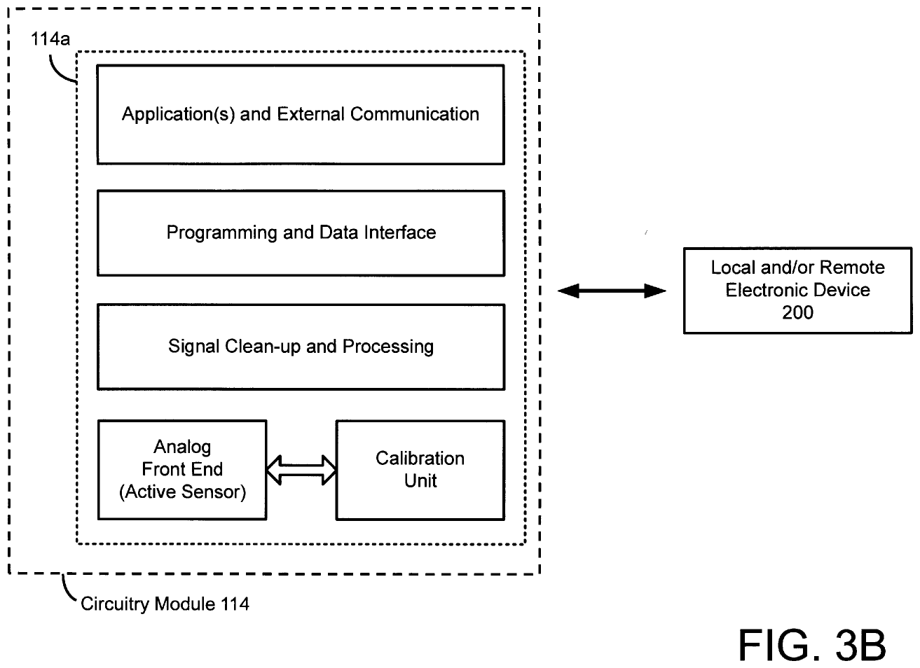

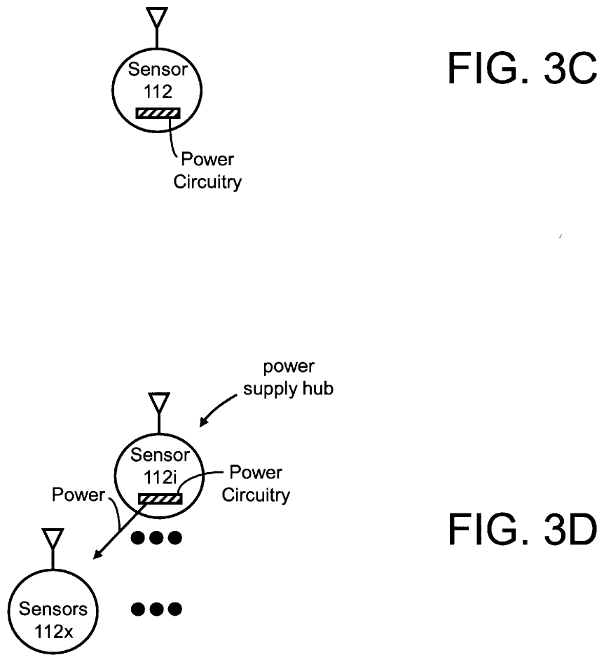

With reference to FIGS. 3A and 3B, in one embodiment, the circuitry module may include a processor, controller circuitry, and transmitter/receiver circuitry. The processor may be configured, in operation, to receive bioimpedance data from the bioimpedance sensors and calculate, assess, determine and/or monitor a fluid state or state of hydration in an animal body (or portion thereof) using the bioimpedance data change(s) in the fluid state or state of hydration in/of the body (or portion thereof). The controller circuitry may control the operation of the bioimpedance sensors, including, for example, control when the sensors acquire bioimpedance data. The transmitter/receiver circuitry, in one embodiment, is configured to transmit control signals to the bioimpedance sensors and receive bioimpedance data from the bioimpedance sensors. As noted above, the communication between the circuitry module and the bioimpedance sensors may employ wired or wireless techniques and, as such, the transmitter/receiver circuitry may include wired and/or wireless circuitry to implement such techniques to support or facilitate the functions/operations of the circuitry module. Notably, the transmitter/receiver circuitry of the circuitry module may provide communications to the portable and/or wearable electronic device (see, e.g., FIG. 2B) and/or the remote circuitry or processing system (see, e.g., FIG. 2D).

With continued reference to FIGS. 3A and 3B, the circuitry module may further include power circuitry to enable the bioimpedance sensors to generate and acquire data which is representative of the bioimpedance of the body (or portion thereof) of the user. Here, the power circuitry, in operation, may provide the bioimpedance sensors, among other things, a DC voltage and/or DC current from which the sensors generate and apply or output an electrical current (e.g., DC current), via an electrode(s) of bioimpedance sensor(s), to the body of the user. The electrodes of the bioimpedance sensors measure a resultant change in voltage. The impedance of the body, or portion thereof, may be derived from the detected change in voltage. In one embodiment, the data acquired by the sensors are provided to the processor to calculate, assess, determine and/or monitor a fluid state or state of hydration in an animal body (or portion thereof) using the bioimpedance data change(s) in the fluid state or state of hydration in/of the body (or portion thereof).

Notably, in one embodiment, one or more, or all of the bioimpedance sensors include power circuitry (e.g., a battery), resident therein, to responsively and controllably provide DC voltage and/or DC current to the electrode(s) to apply or output an electrical current (e.g., DC current), via the electrode(s) of bioimpedance sensor(s), to the body of the user. (See FIG. 3C). In this embodiment, the bioimpedance sensors include the power circuitry (as compared to receiving the power from the circuitry module). In yet another embodiment, one or more bioimpedance sensors is/are connected to power circuitry (e.g., a battery), of an associated bioimpedance sensor that provides the connected bioimpedance sensor(s) a DC voltage and/or DC current from which to generate and apply or output an electrical current (e.g., DC current), via an electrode(s) of the connected bioimpedance sensor(s), to the body of the user. (See FIG. 3D). In this embodiment, one or more bioimpedance sensors (sensor 112i) function or operate as a power supply hub for associated bioimpedance sensor(s) (sensor 112x, . . . ) that (i) receive power from the bioimpedance sensor having the power supply hub and (ii) generate and apply/output an electrical current (e.g., DC current) via an electrode(s) to the body of the user. Notably, the bioimpedance sensor embodiments described and illustrated in connection with FIGS. 3C and 3D may be implemented in any of the embodiments set forth herein (e.g., the embodiment illustrated in FIG. 1A).

With reference to FIGS. 3A and 3B, in the embodiment, the circuitry module may further include an application(s) and external communications submodule, a programming and data interface submodule, a signal processing submodule, a calibration unit (e.g., with IC/SOC (integrated circuit/system-on-chip) components programmed to execute firmware/software, and an analog front end that receives the signals from the sensors. The programming and data interface submodule may include a graphical user interface that allows for programming of threshold settings, which may be selected at a group level (such as wearers falling into a particular demographic group or activity type) or personal level, and the ability to dynamically alter the settings. For example, data across various subjects may be collected and analyzed/annotated by demographic, environmental, dietary, medication data, etc. to inform a base line setting for an individual falling into such group. In turn, the measurements for each individual may further inform how group threshold levels should be set. Alternatively or in addition to the above, programming and threshold settings/calibrations may be input from a remote source or when circuitry module 114 is communicatively coupled to a remote source, such as via a dock, transmission line, or wirelessly when circuitry module 114 is not in use in wearable sensing garment 100.

In various embodiments, the programming and data interface submodule include the processor, suitably programmed, for example, to implement closed-loop program instructions configured to enable autonomous functions and applications (e.g., artificial intelligence and/or machine learning (“AI/ML”)), derive personal models for settings based on memory storage and past activity, and/or detected environmental or physiological properties. The communications interface may further comprise one or more feedback mechanisms (audio, haptic and/or visual) to alert a user/wearer of wearable sensing garment of a hydration state, whether satisfactory or undesirable. As noted, such feedback may be audible, visible, and/or tactile (e.g., vibration, buzzing, a beep, a light flashing, etc.).

In one embodiment, circuitry module 114 contains an outgoing calibrated current source (e.g., a variable DC current source or a modulated current source), such as for example, a rechargeable battery or replaceable. Thus, a small current may be sent by the electrode(s) of a bioimpedance sensor(s) and a responsive change in voltage (magnitude and/or phase), due to impedance, is measured. The signal processing submodule may include an amplifier for the return signal, a processor programmed with motion artifact suppression algorithms, and/or AI/ML subroutines.

With particular reference to FIG. 3B, the power circuitry of circuitry module 114 may include a battery, which may be rechargeable and/or replaceable, to power the circuitry module. In this way, the circuitry module is portability. Further, the external communication submodule (which includes the transmitter/receiver circuitry) may include a relatively wireless transmitter (e.g., low energy such as low power Bluetooth) cellular based technology and/or a radio transmitter that transmits the processed signal, for example to a remote location, which may be a computing device such as a smartphone, smartwatch, tablet, or other computing device, or multiple such devices and configured to, for example, transmit (e.g., continuously) sensed data from sensing electrodes 112 (see, e.g., FIGS. 2B and 3B). The circuitry module 114 may also include one or more data and/or electrical communication ports so as to provide the ability to transmit data and/or power via a wired architectures.

In the event that electrodes of one or more bioimpedance sensors lose contact to the body of the user (whether intermittently or permanently/continuously) due to, for example, body motion or other factors, the processor (of the signal processing submodule) may be configured to disregard/ignore data acquired from such sensors and employ only “valid” data from the bioimpedance sensors that include sufficient contact to the body of the user. In one embodiment, the processor may be programmed with AI/ML subroutines configured to analyze continuous segments of the data stream from those sensors that include sufficient contact to the body of the user and confine analysis to data that is determined to meet threshold quality levels (i.e., those sensors that include sufficient contact to the body of the user).

Notably, in one embodiment, the sensors of bioimpedance sensor network(s) may be augmented or supplemented by the addition of other bioimpedance sensors and/or other types of sensors, both electrical and chemical, including, for example, continuous blood pressure via photoplethysmography (PPG), sweat analysis, glucose by bioimpedance, pulse oximetry, body temperature, and combinations thereof.

In those embodiment where circuitry module 114 includes a processor, the processor may include, for example, one or more of a processors of any kind or type, a system-on-chip (SoC), and dedicated hardware (e.g., an application specific integrated circuit, a field programmable gate array, a complex programmable logic device, and other similar dedicated hardware structures, or a combination thereof). The processor may be suitable programmed to implement the functions and operations described here. The program(s) may be stored in memory in the circuitry module. (See, FIG. 3A).

Together with the memory that stores instructions executable by the processor, the processor and instructions may be configured to perform the various operations described herein.

The number and particular bioimpedance sensors that are incorporated in and/or form the bioimpedance sensor network(s) may be fixed and/or programmable (more than one-time programmable). For example, in one embodiment, certain of the bioimpedance sensor network(s) may be fixed (e.g., at manufacture, the bioimpedance sensor(s) that form the network(s) is/are fixed) and certain other bioimpedance sensor network(s) may be programmable (e.g., the bioimpedance sensor(s) that form such network(s) is/are programmable (e.g., more than one-time programmable) at start-up and/or during operation of the sensing garment). In another embodiment, bioimpedance sensor network(s), in relation to the number and particular bioimpedance sensors, are fixed (e.g., at start-up or initialization). In yet another embodiment, bioimpedance sensor network(s), in relation to the number and particular bioimpedance sensors, are programmable (i.e., more than one-time programmable), for example, in situ.

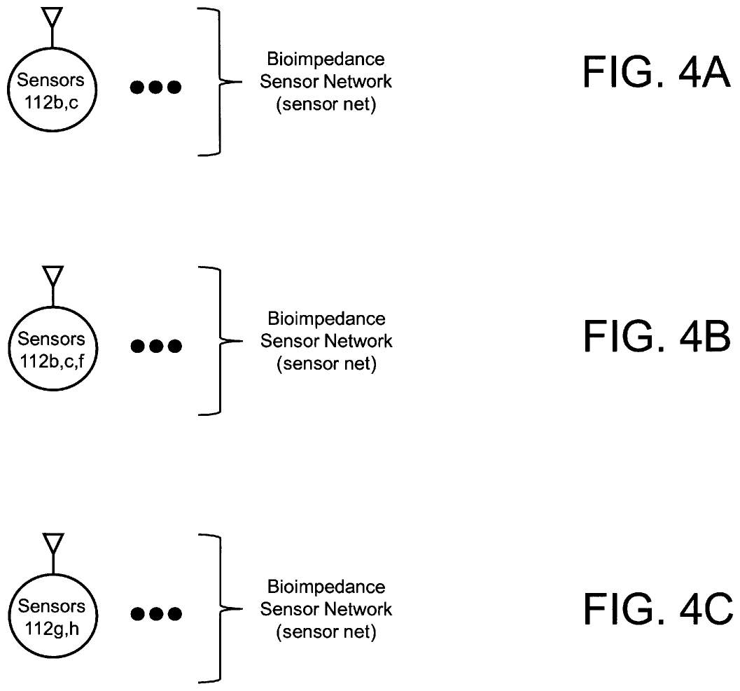

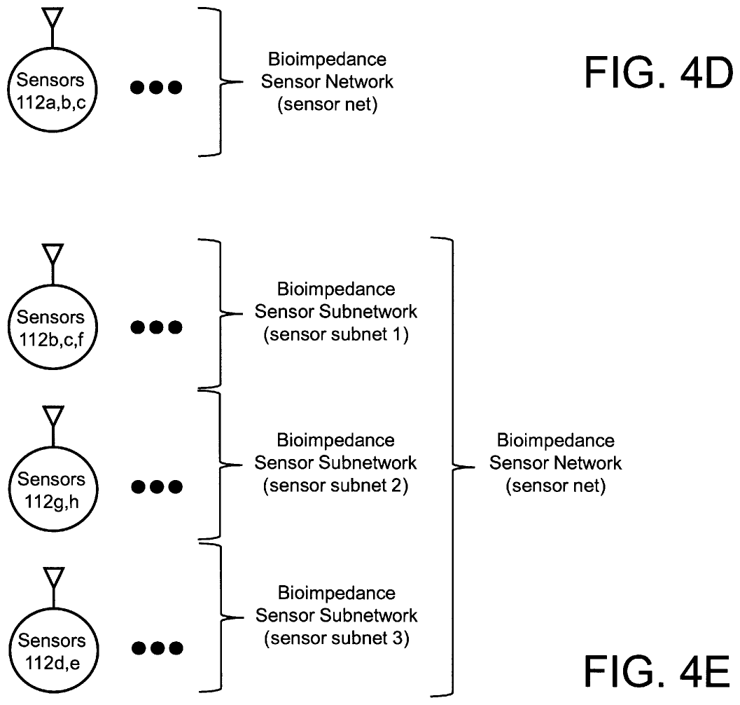

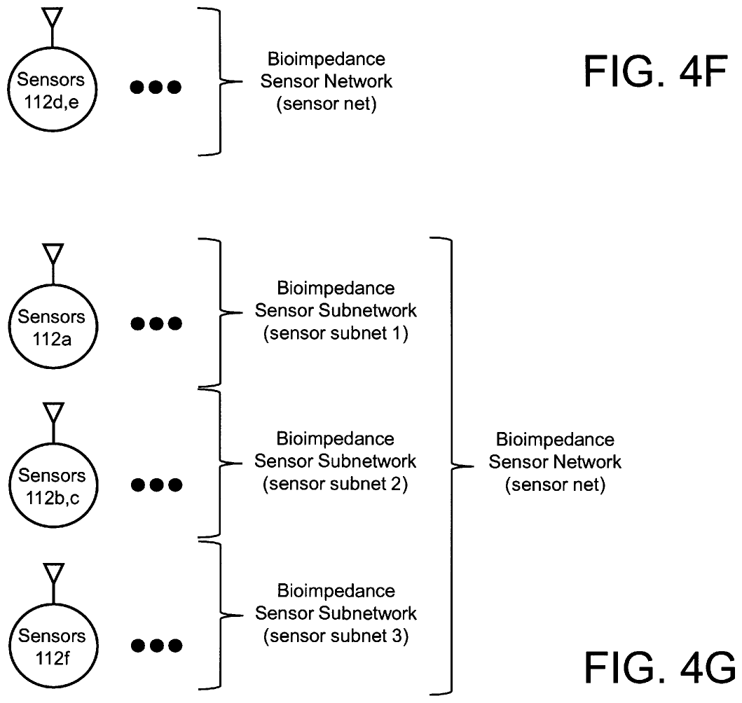

With reference to FIGS. 1A and 4A-4G, bioimpedance sensor network 110 may include one or more different combinations of bioimpedance sensors 112x that may be fixed or programmable (more than one-time programmable). In one embodiment, a sensor network includes bioimpedance sensors 112b and 112c to sense, acquire, detect and/or measure bioimpedance data (magnitude and/or phase data) to, in one embodiment, calculate, assess, determine and/or monitor data associated with, corresponding to and/or representative of a biological properties (e.g., fluid state or state of hydration, or changes therein) in/of a user. (See FIG. 4A). Notably, the network of bioimpedance sensors 112b and 112c may be a subnetwork or the only sensor network employed to measure and/or acquire bioimpedance data. In one embodiment, the bioimpedance sensor 112f may be included, incorporated and/or integrated (e.g., at manufacture or in situ) into the network of bioimpedance sensors 112b and 112c to provide or form a new bioimpedance sensor network 110. (See FIG. 4B). The bioimpedance sensor networks 110 of FIGS. 4A and 4B may be advantageous to employ in connection with monitoring and/or detection of a fluid state (e.g., fluid retention), or change therein, in the chest or abdomen region of the user or wearer of the sensing garment 100, which may signal a variety of health conditions, including heart conditions, pulmonary conditions, cardio-pulmonary conditions, whereas fluid retention in the abdomen region may indicate intestinal conditions, swelling, and/or kidney conditions.

With reference to FIG. 4C, in another embodiment, the bioimpedance sensor network of sensors 112g and 112h may include a subnetwork or the only sensors employed to measure and/or acquire bioimpedance data. In yet another embodiment, bioimpedance sensor network 110 includes three bioimpedance sensor subnetworks including a first bioimpedance sensor subnetwork including sensors 112b, 112c and 112f (corresponding to the chest and abdomen), a second bioimpedance sensor subnetwork including sensors 112g and 112h (corresponding to the legs), and a third bioimpedance sensor subnetwork including sensors 112d and 112e (corresponding to the arms). (See FIG. 4E). The bioimpedance sensors of the sensor network and subnetworks are organized and arranged according to regions of the body—and the body of the user may be viewed as being compartmentalized or segmented, from a data acquisition and analysis perspective. Indeed, in these exemplary embodiments, the data processing implemented by the processor (whether the processors is local or remote) may organize data processing on a subnetwork basis (all of the sensors of a given network in the system) or on a network basis (all of the sensors in the system) to, for example, improve and/or focus determination, calculation and/or monitoring of biological properties (e.g., fluid state or state of hydration, or changes therein) of selected regions of the body in/of a user with greater accuracy.

Notably, as intimated herein, in any or all of the embodiments, the processor may also employ user information (e.g., height, age, and gender), to improve accuracy of monitoring, assessment and/or determination of biological properties (e.g., fluid state or state of hydration, or changes therein).

With reference to FIG. 1A, wearable sensing garment 100 may include or integrate a sensor network include any number of bioimpedance sensors—albeit eight sensors 112a-112h are illustrated in one embodiment. More or less sensors could be incorporated in a variety of manners, as has been described above, so as to be relatively easily adorned on a wearer's body and so as to allow the wearer to go about various day-to-day and even strenuous, more demanding activities, while relatively continuously measuring the bioimpedance of the wearer and processing the measured values to monitor for a fluid state of the wearer. Moreover, it is envisioned as within the scope of the present disclosure that circuitry module 114 may be programmed to selectively receive data from fewer than all of the bioimpedance sensor and/or employ data from less than all of the sensors included in/on wearable sensing garment 100 or in the sensor network or subnetwork. In this way, the processor may change or modify (e.g., during operation) the sensor network and/or subnetwork(s)—for example, based on a body type of a wearer and/or a condition being monitored.

There are many inventions described and illustrated herein. While certain embodiments, features, attributes and advantages of the inventions have been described and illustrated, it should be understood that many others, as well as different and/or similar embodiments, features, attributes and advantages of the present inventions, are apparent from the description and illustrations. As such, the above embodiments of the inventions are merely exemplary. They are not intended to be exhaustive or to limit the inventions to the precise forms, techniques, materials and/or configurations disclosed. Many modifications and variations are possible in light of this disclosure. It is to be understood that other embodiments may be utilized and operational changes may be made without departing from the scope of the present inventions. As such, the scope of the inventions is not limited solely to the description above because the description of the above embodiments has been presented for the purposes of illustration and description.

For example, although the present inventions have been largely/extensively described in the context of a sensing wearable garment, as stated above, one or more bioimpedance sensors may be directly attached to the skin of a user's body. For example, bioimpedance sensors may be attached directly to the body (e.g., the skin) via adhesive, medical tape, or a lightweight harness that strings together one or more bioimpedance sensors, or a combination of any of the above. These embodiments may be employed in lieu of a wearable sensing garment or accessory (i.e., one or more wearable accessories, such as a belt, ring, watch, necklace, collar, bracelet, etc.), or in addition thereto. All combinations and permutations are intended to fall within the scope of the present inventions

Moreover, various aspects of the present inventions contemplate one or more of multiple bioimpedance sensors forming a network (sensor net or sensor network) on/around a wearer's body; each sensor by itself and all applied sensors in conjunction delivering individual and composite metrics of bioimpedance, on, for example, a continuous basis to achieve real-time monitoring and feedback; patient-specific, context-specific dynamic threshold/trigger values for impaired fluid state determination; recovery and interpolation of measurements, in the event of loss of contact or other intermittent signal situations which impact the integrity of the measured impedance data; export of the sensor network measurements/signal processing as a controllable ‘device’ to other applications/programs; export of the raw signals, bioimpedance or any related intermediate output or any related derivative transform of the data to other applications/programs; derivation of patient-specific, context-specific models which may be exported as a program to other applications/programs.

As discussed above, in certain embodiments, a plurality of bioimpedance sensors are integrated into a wearable garment and configured to measure bioimpedance for monitoring a fluid state of a body and configured to be relatively easily adorned by the body so as to permit real-time monitoring of a wearer of the one or more sensors while the human, for example, performs typical day-to-day activities in the wearable sensing garment—in situ (i.e., that is, worn during normal performance or operation of, for example, a human, and/or worn in the expected/normal environment of performance or operation of, for example, the human). Various embodiments contemplate integration of a bioimpedance sensor network including multiple sensors connected to a circuitry module that is common to the bioimpedance sensors, into a practical wearable garment, such as for example a soldier's uniform, a firefighter's protective garb, an athlete's uniform, or any of a variety of other wearable garments, including but not limited to a garment to be applied to patients in the field by emergency workers and rescuers, as well as in hospitals and other medical facilities. In conjunction with monitoring a fluid state of a body, the circuitry module and/or other circuitry (e.g., portable and/or wearable electronic device and/or remote circuitry (e.g., cloud or edge circuitry such as a server and storage in the “cloud”) using the sensed data to monitor for a variety of medical conditions associated with either undesirable fluid loss (e.g., dehydration) or undesirable fluid retention (e.g., heart conditions, cardio-pulmonary conditions, pulmonary conditions, kidney conditions, intestinal conditions, general swelling/circulation conditions, and/or wound conditions).

In addition, various embodiments further contemplate substantially continuous bio-impedance sensing at various points of the body simultaneously, permitting a robust determination of the fluid state of a wearer of the garment comprising the sensor network. Further, various embodiments utilize sensors configured to measure bioimpedance to sense a fluid state of a body, either generally throughout the body and/or at specific locations that may be more associated with particular medical conditions.

As noted above, the wearable sensing garment and/or accessory (having bioimpedance sensors) may be a full body suit such as a war fighter's or fire fighter's gear, a vest that would cover regions of the body associated with the thorax, a vest or long sleeve shirt portion with separate anklets garments containing the sensors by the ankles with no sensors in the pant portion, or any other combination or permutation of garment. Thus, the wearable sensing garment may be one contiguous item (e.g., a body suit) or a plurality of discrete items including separate/distinct or integrated garment sections corresponding to one or more portions of the body of the animal—whether the garment sections are to be worn concurrently and/or separately; all combinations or permutations thereof are intended to fall within the scope of the present inventions.

Although not fully illustrated, in one embodiment, one or more, or all of bioimpedance sensor(s) illustrated in the exemplary wearable sensing garment 100 of FIGS. 1A and 1B may represent a plurality of bioimpedance sensors disposed on/in the associated region (e.g., a plurality of bioimpedance sensors (e.g., 2, 3, 4, 5, 6, etc.) located in/on each of leg (112g/112h), each arm (112d/112e), the chest region (112b/112c), abdomen region (112f) and/or neck area (112a)). The electrode(s) of such sensors may be located on one, or more (or all) sides of the associated region in order to obtain data from different areas of the region. All combinations and permutations of bioimpedance sensors disposed on/in a particular region, and electrode location(s) in that region, are intended to fall within the scope of the present inventions. Moreover, such embodiments may be implemented in connection with those embodiments where the sensors are located directly on the body of the user (e.g., the skin of the user's body).

In accordance with various embodiments, the sensor network may be integrated into the garment by placing the sensors at various positions so as to sense data from over the entire body and/or in zones, which may be selected depending on application. For example, one or more sensors may be positioned to sensed data in the abdomen or chest area in the case of monitoring for dehydration or monitoring for heart failure and/or pneumonia, respectively. As another example, one or more sensors may be placed at a location of a body that is at risk of undesirable fluid retention, such as at a wound and/or insect bite site. In general, where one or more bioimpedance sensors are located on a body and/or how they are integrated into a garment or other wearable accessory and/or direct means of application may be selected based on a how the sensors are intended to be use and under what conditions.

In accordance with various other embodiments, the present disclosure contemplates the addition of other sensor types into a wearable garment, accessory, or direct application to a body, so as to add other measurements to the collected data stream, which may thereby increase the diagnostic sensitivity and specificity of the information sensed and medical relevance. For instance, the combination of low body fluids, low blood pressure, and high body temperature together would be expected to be more diagnostic than body fluid content alone. In one embodiment, a wearable motion corrected EKG sensor may be incorporated along with the one or more bioimpedance sensors and communicatively coupled to the circuitry module monitor for cardiac rhythm detection and provide sensed data associated with the same to the data stream transmitted to the circuitry module. Real-time pulse oximetry may also be sensed, which, for example, may have particular application in the fire-fighting context to monitor for inhalation of smoke. Those having ordinary skill in the art would appreciate how various other sensors could be selected and used in conjunction with the bioimpedance sensors described herein to monitor for specific medical conditions.

Notably, as intimated herein, in any or all of the embodiments, the processor may also employ user information (e.g., height, age, and gender), to improve accuracy of assessment or determination of biological properties (e.g., fluid state or state of hydration, or changes therein).

The processed data may be transmitted by the wearable sensing garment either directly to a remote circuitry in, for example, a central station (e.g., in the cloud—see FIG. 2D) for analysis and action (e.g., implement preventive measures, corrective intervention and/or determination of appropriate treatment) and/or a smartphone, smart watch, and/or other similar technology configured for health monitoring, data collection and/or implementation of preventive measures, corrective intervention and/or determination of appropriate treatment (see FIGS. 2A-2C).

Notably, the term “circuitry”, means, among other things, a circuit (whether integrated or otherwise), a group of such circuits, one or more processors, one or more state machines, one or more processors implementing software, one or more gate arrays, programmable gate arrays and/or field programmable gate arrays, or a combination of one or more circuits (whether integrated or otherwise), one or more state machines, one or more processors, one or more processors implementing software, one or more gate arrays, programmable gate arrays and/or field programmable gate arrays.

Importantly, the present inventions are neither limited to any single aspect nor embodiment thereof, nor to any combinations and/or permutations of such aspects and/or embodiments. Moreover, each of the aspects of the present inventions, and/or embodiments thereof, may be employed alone or in combination with one or more of the other aspects of the present inventions and/or embodiments thereof.

Notably, reference herein to “one embodiment” or “an embodiment” (or the like) means that a particular feature, structure, or characteristic described in connection with the embodiment may be included, employed and/or incorporated in one, some or all of the embodiments of the present inventions. The usages or appearances of the phrase “in one embodiment” or “in another embodiment” (or the like) in the specification are not referring to the same embodiment, nor are separate or alternative embodiments necessarily mutually exclusive of one or more other embodiments, nor limited to a single exclusive embodiment. The same applies to the term “implementation.” The present inventions are neither limited to any single aspect nor embodiment thereof, nor to any combinations and/or permutations of such aspects and/or embodiments. Moreover, each of the aspects of the present inventions, and/or embodiments thereof, may be employed alone or in combination with one or more of the other aspects of the present inventions and/or embodiments thereof. For the sake of brevity, certain permutations and combinations are not discussed and/or illustrated separately herein.

Further, an embodiment or implementation described herein as “exemplary” is not to be construed as ideal, preferred or advantageous, for example, over other embodiments or implementations; rather, it is intended convey or indicate the embodiment or embodiments are example embodiment(s).

Although the present inventions have been described in certain specific aspects, many additional modifications and variations would be apparent to those skilled in the art. It is therefore to be understood that the present inventions may be practiced otherwise than specifically described without departing from the scope and spirit of the present inventions. Thus, embodiments of the present inventions should be considered in all respects as illustrative/exemplary and not restrictive.

The terms “comprises,” “comprising,” “includes,” “including,” “have,” and “having” or any other variation thereof, are intended to cover a non-exclusive inclusion, such that a process, method, circuit, system, article, or apparatus that comprises a list of parts or elements does not include only those parts or elements but may include other parts or elements not expressly listed or inherent to such process, method, article, or apparatus. Further, use of the terms “connect”, “connected”, “connecting” or “connection” herein should be broadly interpreted to include direct or indirect (e.g., via one or more conductors and/or intermediate devices/elements (active or passive) and/or via inductive or capacitive coupling)) unless intended otherwise (e.g., use of the terms “directly connect” or “directly connected”).

The terms “a” and “an” herein do not denote a limitation of quantity, but rather denote the presence of at least one of the referenced item. Further, the terms “first,” “second,” and the like, herein do not denote any order, quantity, or importance, but rather are used to distinguish one element/circuit/feature from another.

In the claims, the term “garment” means or includes “garment”, “clothing” and/or “accessory” unless expressly stated to the contrary; for the avoidance of doubt, the same is true of the plural forms thereof. In addition, in the claims, the term “state” (e.g., “fluid state of a body”) means or includes “state” and/or “status” (e.g., or “fluid state in a body” and/or “fluid status in a body”); for the avoidance of doubt, the same is true of the plural forms thereof. Further in the claims, the term “on the garment” means or includes “on the garment” and “in the garment.”

Again, there are many inventions described and illustrated herein. While certain embodiments, features, attributes and advantages of the inventions have been described and illustrated, it should be understood that many others, as well as different and/or similar embodiments, features, attributes and advantages of the present inventions, are apparent from the description and illustrations.

What is claimed is:

1. A bioimpedance sensing device to measure bioimpedance data of a body of a user and determine a fluid state thereof, the device comprising:
wearable sensing garment to, in operation, be worn on the body by the user in situ, the wearable sensing garment including:
a plurality of bioimpedance sensors disposed on the wearable sensing garment wherein each bioimpedance sensor:
is located at a unique spatial location on the garment and is configured to contact a body of the user that corresponds to the unique spatial location of the associated bioimpedance sensor, and
in operation, measures bioimpedance data at the unique spatial location of the associated bioimpedance sensor; and
a circuitry module physically coupled to the body of the user and configured to be communicatively coupled to the plurality of bioimpedance sensors, wherein the circuitry module includes:
communication circuitry, coupled to the processor, to receive the measured bioimpedance data from the plurality of bioimpedance sensors,
a processor configured, in operation, to process the measured bioimpedance data to determine the fluid state of the body of the user, or a change therein, and
a battery to power the circuitry module; and
wherein, in operation, the plurality of bioimpedance sensors are configured to transmit the measured bioimpedance data to the circuitry module.

2. The bioimpedance sensing device of claim 1 wherein:
the circuitry module further includes power circuitry, coupled to the plurality of bioimpedance sensors, to provide power to the plurality of bioimpedance sensors.

3. The bioimpedance sensing device of claim 1 wherein:
the wearable sensing garment is configured to fit tightly to the body of the user.

4. The bioimpedance sensing device of claim 1 wherein:
the circuitry module further includes power circuitry, wirelessly coupled to the plurality of bioimpedance sensors, to provide power to the plurality of bioimpedance sensors.

5. The bioimpedance sensing device of claim 1 wherein:
the wearable sensing garment consists essentially of a shirt or vest that is configured to fit tightly to a chest and an abdomen of the user, the plurality of bioimpedance sensors, the circuitry module, and wiring to connect the plurality of bioimpedance sensors to the circuitry module.

6. The bioimpedance sensing device of claim 5 wherein:
the wearable sensing garment further includes a pouch or pocket to receive the circuitry module.

7. The bioimpedance sensing device of claim 1 wherein:
the plurality of sensors are respectively placed at the multiple locations on the wearable sensing garment so as to correspond to different locations on a chest, an abdomen locations, and limbs of the body of the user.

8. The bioimpedance sensing device of claim 1 wherein:
the wearable sensing garment is configured to fit tightly to the body of the user such that the plurality of sensors substantially continuously contact the body of the user in situ.

9. The bioimpedance sensing device of claim 1 wherein:
the processor of the circuitry module is configured to determine a change in state of hydration of the body of the user using the measured bioimpedance data.

10. A bioimpedance sensing device to measure bioimpedance data of a body of a user, the device comprising:
wearable sensing garment to, in operation, be worn on the body by the user in situ, the wearable sensing garment including:
a first garment including a shirt or vest that is configured to fit to a chest and an abdomen of the user,
a second garment including long pants to fit to each leg of the user,
a first plurality of bioimpedance sensors disposed on the first garment wherein each bioimpedance sensor of the first plurality of bioimpedance sensors:
is located at a unique spatial location on the first garment and is configured to contact a location on the chest and the abdomen of the user that corresponds to the unique spatial location of the associated bioimpedance sensor, and
in operation, measures bioimpedance data at the unique spatial location of the associated bioimpedance sensor;
a second plurality of bioimpedance sensors disposed on the second garment wherein each bioimpedance sensor of the second plurality of bioimpedance sensors:
is located at a unique spatial location on the second garment and is configured to contact a location on one of the legs of the user that corresponds to the unique spatial location of the associated bioimpedance sensor, and
in operation, measures bioimpedance data at the unique spatial location of the associated bioimpedance sensor;
a circuitry module physically coupled to the body of the user and configured to be communicatively coupled to the first and second plurality of bioimpedance sensors, wherein the circuitry module includes:
communication circuitry, coupled to the processor, to receive the measured bioimpedance data from the first and second plurality of bioimpedance sensors, and
a processor configured, in operation, to process the measured bioimpedance data, and
wherein, in operation, the plurality of sensors are configured to transmit the measured bioimpedance data to the circuitry module.

11. The bioimpedance sensing device of claim 10 wherein:
the circuitry module further includes power circuitry, first and second plurality of bioimpedance sensors, to provide power to the first and second plurality of bioimpedance sensors.

12. The bioimpedance sensing device of claim 10 wherein:
the first garment of the wearable sensing garment is configured to fit tightly to the chest and abdomen of the user, and
the second garment of the wearable sensing garment is configured to fit tightly to the legs of the user.

13. The bioimpedance sensing device of claim 10 wherein:
the communication circuitry of the circuitry module wirelessly coupled to the first and second plurality of bioimpedance sensors, to wirelessly receive the measured bioimpedance data from the first and second plurality of bioimpedance sensors.

14. The bioimpedance sensing device of claim 10 wherein:
the circuitry module further includes power circuitry, wirelessly coupled to the first and second plurality of bioimpedance sensors, to provide power to the first and second plurality of bioimpedance sensors.

15. The bioimpedance sensing device of claim 10 wherein:
the circuitry module physically couples to the wearable sensing garment.

16. The bioimpedance sensing device of claim 10 wherein:
the processor of the circuitry module is configured to process the measured bioimpedance data from the first and second plurality of bioimpedance sensors to determine the fluid state of the body of the user, or a change therein.

17. The bioimpedance sensing device of claim 10 wherein:
the processor of the circuitry module is configured to determine a change in state of hydration of the body of the user using the measured bioimpedance data from the first and second plurality of bioimpedance sensors.

18. A bioimpedance sensing device to measure bioimpedance data of a body of a user and determine a fluid state thereof, the device comprising:
wearable sensing garment to, in operation, be worn on the body by the user in situ, the wearable sensing garment including:
a plurality of bioimpedance sensors disposed on the wearable sensing garment wherein each bioimpedance sensor:
is located at a unique spatial location on the wearable sensing garment and is configured to contact a body of the user that corresponds to the unique spatial location of the associated bioimpedance sensor, and
in operation, measures bioimpedance data at the unique spatial location of the associated bioimpedance sensor; and
a circuitry module configured to be communicatively coupled to the plurality of bioimpedance sensors, wherein the circuitry module includes:
communication circuitry, coupled to the processor, to receive the measured bioimpedance data from the plurality of bioimpedance sensors, and
a processor configured, in operation, to process the measured bioimpedance data to determine the fluid state of the body of the user, or a change therein; and
wherein, in operation, the plurality of bioimpedance sensors are configured to transmit the measured bioimpedance data to the circuitry module while worn by the user in situ.

19. The bioimpedance sensing device of claim 18 wherein:
the plurality of sensors are respectively placed at the multiple locations on the wearable sensing garment so as to correspond to different locations on a chest and abdomen locations of the user.

20. The bioimpedance sensing device of claim 19 wherein:
the wearable sensing garment is configured to fit tightly to a chest and an abdomen of the user.
